(19) United States
(12) Patent Application Publication
(10) Pub. No.: US 2006/0106321 A1
(43) Pub. Date: May 18, 2006

(54) Device, system, and method for detecting, localizing, and characterizing plaque-induced stenosis of a blood vessel

(75) Inventors: Reuven Lewinsky, Caesaria (IL); Nir Berzak, Zikhron-Yaakov (IL); Uri Amir, Or Yehuda (IL); Mordechai Bliweis, Haifa (IL)

Correspondence Address: Martin Moynihan;Anthony Castorina, Suite 207, 2001 Jefferson Davis Highway, Arlington, VA, 22202, US

(73) Assignee: Galil Medical Ltd., Yokneam (IL)

(21) Appl. No.: 10/542,387

(22) PCT Filed: Jan. 15, 2004
(86) PCT No.: PCT/IL04/00046

§ 371 Date: Jan. 10, 2006

Related U.S. Application Data
(60) Provisional application No. 60/440,361, filed on Jan. 16, 2003.

(51) Int. Cl. A61B 5/02

(52) U.S. Cl. 600/491

(57) ABSTRACT

The present invention is of system, device, and method for detection, localization, and characterization of plaque-induced stenosis of a blood vessel. More particularly, the present invention relates to a balloon catheter having an expandable balloon insertable into a blood vessel, which balloon comprises a plurality of pressure sensors operable to detect stenosis of the vessel, and further operable to report degrees of compressibility of stenotic regions of plaque within the vessel, thereby distinguishing between standard and vulnerable plaque.

### FIELD AND BACKGROUND OF THE INVENTION

The present invention relates to devices and methods for detection, localization, and characterization of plaque-induced stenosis of a blood vessel. More particularly, the present invention relates to a balloon catheter having an expandable balloon insertable into a blood vessel, which balloon comprises a plurality of pressure sensors operable to detect stenosis of the vessel, and further operable to report degrees of compressibility of stenotic regions of plaque within the vessel, thereby distinguishing between standard and vulnerable plaque.

Most adults suffer to some degree from atherosclerotic plaque within blood vessels of the body. Plaque may limit blood flow through the vessel, causing dangerous tissue degeneration in extreme cases. Stenosis caused by plaque is often responsible for ischemic heart disease. The presence of plaque in blood vessels may also lead to thrombosis, endangering heart, lung, and brain tissue in particular.

Percutaneous transluminal angioplasty (PTA) is a treatment of choice for most stenotic conditions. In PTA, an inflatable balloon catheter or similar device is used to dilate a stenotic region of a blood vessel, thereby facilitating blood flow through the affected region. Various alternative and/or complementary procedures are used in treatment of stenotic conditions. These include arthrectomy, laser angioplasty, the use of stents, and the use of cryosurgical techniques to cool affected regions during or following compression of an affected area by angioplasty balloon.

The effectiveness of the above treatment methodologies is highly dependent on correct diagnostic localization of the areas to be treated. Yet, stenotic areas are, by their nature, not easily observable. A variety of strategies for locating regions of plaque within a blood vessel, and for characterizing that plaque, have been proposed and tested. Joye et al., in U.S. Pat. No. 6,602,246, teaches methods based on differential temperature readings from within a blood vessel, in recognition of the fact that the type of plaque particularly prone to create thromboses, termed “vulnerable plaque”, tends to be inflamed and therefore is at a higher temperature than standard stenotic plaque and normal healthy vascular tissue. Joye also lists angiography, intravascular ultrasound, angioscopy, magnetic resonance imaging, magnetic resonance diffusion imaging; spectroscopy, infrared spectroscopy, scintigraphy, optical coherence tomography, electron beam computed tomographic scanning, and thermography as prior art methods which have been used, with varying success, to locate regions of plaque within a vessel.

None of the above methods, however, has been found to be entirely successful, and most are complex and expensive. Thus there is a widely felt need for, and it would be advantageous to have, a device and method for locating and characterizing stenotic regions within a blood vessel, which device and method are relatively simple in construction and use, and relatively inexpensive.

Plaque may be characterized as belonging to one of two general types, “standard” stenotic plaque, presenting relatively little risk of thromboses, and “vulnerable” plaque, presenting a high thrombotic risk. Distinguishing between these two types of plaque, when examining a stenotic region of a vessel, is an important diagnostic goal, since both prognosis and recommended treatment differs: a procedure which may be adequate or even optimal for treating standard plaque may be inappropriate and even dangerous if used to treat vulnerable plaque. Hence, there is a widely felt need for, and it would be advantageous to have, a device and method for distinguishing between standard and vulnerable plaque, which device and method are relatively simple to construct and to use, and relatively inexpensive.

### SUMMARY OF THE INVENTION

According to one aspect of the present invention there is provided a balloon catheter operable to detect obstruction of blood flow within a blood vessel, comprising:

a. a controllably inflatable balloon;

b. a first pressure sensor operable to measure and report ambient pressure within the blood vessel at a position proximal to the balloon; and

c. a second pressure sensor operable to measure and report ambient pressure within the blood vessel at a position distal to the balloon.

According to further features in preferred embodiments of the invention described below, at least one of the first and second pressure sensors is operable to report pressure measurements to a data receiver by wire connection, or by wireless connection.

According to another aspect of the present invention there is provided a method for detecting obstruction of blood flow within a blood vessel, comprising:

a. introducing into the blood vessel a balloon catheter which comprises

i. a balloon operable to be controllably inflated under pressure of a pressurized inflating fluid,

ii. a first pressure sensor operable to report ambient pressure within the blood vessel at a position proximal to the balloon, and

iii. a second pressure sensor operable to measure and report ambient pressure within the blood vessel at a position distal to the balloon;

b. obtaining a first pressure measurement of ambient pressure at the first sensor;

c. obtaining a second pressure measurement of ambient pressure at the second sensor; and

d. reporting obstruction of blood flow within the vessel if a significant difference is found to exist between the first pressure measurement and the second pressure measurement.

According to further features in preferred embodiments of the invention, a difference between the first pressure measurement and the second pressure measurement is treated as significant if the difference exceeds a predetermined value.

According to still further features in preferred embodiments of the invention, the method further comprises determining a position of a detected obstruction by determining a position of the balloon when a significant difference is found to exist between the first pressure measurement and the second pressure measurement. Position of the balloon may be determined by determining a length of penetration of the catheter in the vessel by reading a graduated scale presented on a proximal portion of the catheter, which scale indicates a length to which the catheter has penetrated into the blood vessel. Alternately, position of the balloon may be determined by utilizing an imaging modality to observe the catheter within the vessel, or by utilizing an imaging modality to observe a marker on the catheter, which marker is visible under the imaging modality. Preferably, the marker is radio-opaque and the imaging modality is a fluoroscope. Alternately, the marker is visible under ultrasound scanning, and the imaging modality is an ultrasound system.

According to yet another aspect of the present invention there is provided a method for measuring an internal dimension of a blood vessel, comprising:

a. introducing into the vessel a balloon catheter having a controllably expandable inflatable balloon and at least one first pressure sensor operable to report pressure between an outer wall of the balloon and an inner wall of the blood vessel;

b. expanding the balloon until contact is established between the outer wall of the balloon and the inner wall of the blood vessel, the contact being indicated by a rise in pressure reported by the at least one first pressure sensor; and

c. determining and reporting an external dimension of the balloon when the rise in pressure is detected, thereby measuring the internal dimension of the blood vessel.

According to further features in the described preferred embodiments, the external dimension of the balloon may be determined by inspecting the balloon under an imaging modality such as an x-ray system or a fluoroscope, or an ultrasound system.

According to still further features in the described preferred embodiments, the external dimension of the balloon is determined by utilizing a second pressure sensor to measure pressure of an inflation fluid inflating the balloon, and calculating the external dimension as a function of the measured pressure of the inflation fluid as reported by the second pressure sensor. The calculation may be based on known characteristics of expansibility of the balloon under varying conditions of pressure.

According to still further features in the described preferred embodiments, the method further comprises utilizing a plurality of the first pressure sensors, which may be arranged in a circumferential configuration on the balloon, or in a plurality of circumferential configurations on the balloon.

According to another aspect of the present invention there is provided a method for distinguishing between standard plaque and vulnerable plaque in a blood vessel, comprising:

a. introducing into the vessel a balloon catheter having a controllably expandable inflatable balloon and at least one first pressure sensor operable to report pressure between an outer wall of the balloon and an inner wall of the blood vessel;

b. expanding the balloon until contact is established between the outer wall of the balloon and the inner wall of the blood vessel, the contact being indicated by a detected rise in pressure reported by the at least one first pressure sensor;

c. further expanding the balloon to a controlled degree;

d. utilizing the at least one first pressure sensor to report pressure between the outer wall of the balloon and the inner wall of the blood vessel;

e. comparing the reported pressure to pressure values appropriate for healthy blood vessel wall tissues;

f. reporting presence of standard plaque if the reported pressure is greater than the values appropriate for healthy blood vessel tissues; and

g. reporting presence of vulnerable plaque if the reported pressure is less than the values appropriate for healthy blood vessel tissues.

The present invention successfully addresses the shortcomings of the presently known configurations by providing a device and method for locating and characterizing stenotic regions within a blood vessel, which device and method are relatively simple to construct and to use, and relatively inexpensive.

The present invention further successfully addresses the shortcomings of the presently known configurations by providing a device and method for distinguishing between standard and vulnerable plaque, which device and method are relatively simple to construct and to use, and relatively inexpensive.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a balloon catheter within a blood vessel, the catheter comprising an expandable balloon and a plurality of pressure sensors, according to an embodiment of the present invention;

FIGS. 2A and 2B are simplified schematics of the balloon catheter of FIG. 1, showing how pressure measurements taken by proximal and distal pressure sensors may be used to diagnose stenosis in a blood vessel, according to ah embodiment of the present invention;

FIG. 3 is a simplified schematic of a preferred embodiment of the present invention, showing a preferred pattern of disposition of a plurality of pressure sensors along and around a balloon catheter, according to an embodiment of the present invention; and

FIG. 4 is a simplified schematic of a system for detecting and characterizing stenotic regions of a blood vessel, according to an embodiment of the present invention.

### DESCRIPTION OF THE PREFERRED EMBODIMENTS

The present invention relates to devices and methods for detection, localization, and diagnostic characterization of regions of plaque within a blood vessel. More particularly, the present invention relates to a balloon catheter which comprises an expandable balloon insertable into a blood vessel, which balloon comprises a plurality of pressure sensors operable to report differential pressures at various positions in and around the balloon. The described catheter can be used to detect stenosis in a blood vessel, to measure the position and extent of the plaque region causing the stenotic condition, and to determine the degree of compressibility of the plaque, thereby distinguishing between standard and vulnerable plaque.

The principles and operation of a diagnostic balloon catheter specialized for detecting, localizing, and characterizing plaque within a blood vessel according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

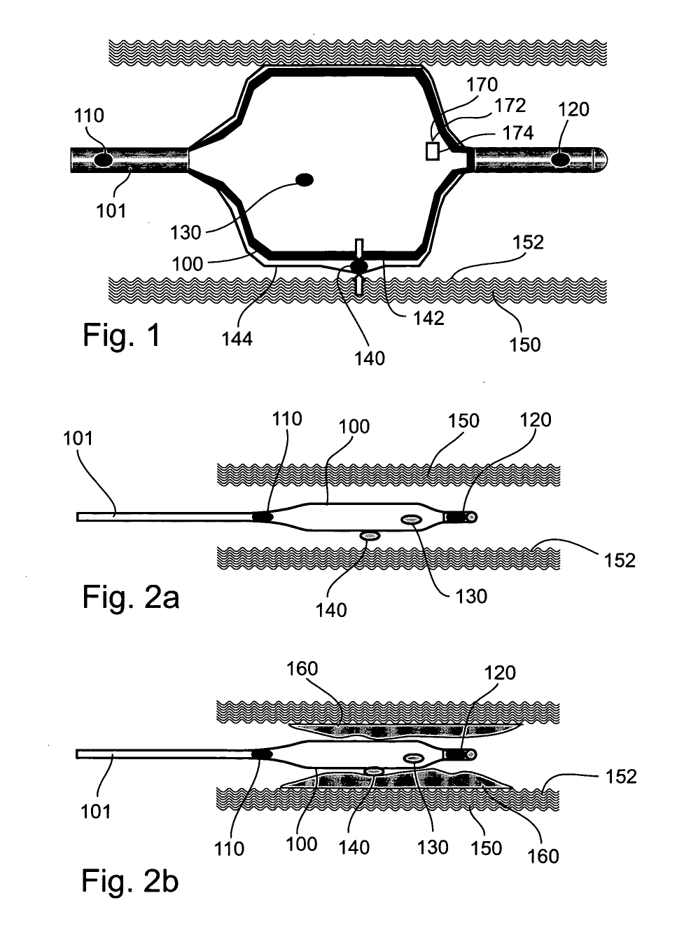

Attention is now drawn to FIG. 1, which presents a simplified schematic of a balloon catheter 101 within a blood vessel 150. Catheter 101 comprises an expandable balloon 100. Balloon 100 is operable to be expanded by inflation by a pressurized fluid delivered to balloon 100 through a pressurized fluid delivery lumen (not shown) in catheter 101.

Catheter 101 further preferably comprises a plurality of pressure sensors 110, 120, 130, and 140.

Pressure sensor 110 is mounted on catheter 101 proximal to balloon 100, or on a proximal portion of balloon 100, and is operable to measure and to report ambient pressure in blood vessel 150 at sensor 110's position, proximal to balloon 100.

Pressure sensor 120 is mounted on catheter 101 at a position distal to balloon 100, or is mounted on a distal portion of balloon 100. Pressure sensor 120 is operable to measure and to report ambient pressure in blood vessel 150 at sensor 120's position, distal to balloon 100.

Optional pressure sensor 130 is mounted within balloon 100, and is operable to measure and to report ambient pressure within balloon 100.

Optional pressure sensor 140 is mounted external to balloon 100. Sensor 140 may be partially embedded in wall 142 of balloon 100, or may be externally attached to or mounted on wall 142 of balloon 100. When balloon 100 is expanded so as to make contact with interior wall 152 of blood vessel 150, sensor 140 is operable to measure and to report pressure between interior wall 152 of blood vessel 150 and exterior wall 142 of expanded balloon 100.

An optional protective sheath 144 may be provided, such that protective sheath 144, rather than sensor 140, comes into direct contact with blood vessel wall 152 of blood vessel 150.

Pressure sensors 110, 120, 130, and 140 may communicate their measurements to a data receiver, such as a data processor, over a wire (e.g., by variation in an electrical resistance as a function of variation in ambient pressure, or by variation in a voltage as a function of variation in ambient pressure), or alternatively, some or all of pressure sensors 110, 120, 130 and 140 may be operable to report measurements to a data receiver by wireless communication.

Attention is now drawn to FIGS. 2A and 2B, each of which is a simplified schematic of balloon catheter 101, shown positioned within a blood vessel 150. FIGS. 2A and 2B serve to show how pressure measurements taken by pressure sensors 110 and 120 may be used to diagnose stenosis in a blood vessel.

FIG. 2A presents catheter 101 within a blood vessel having no stenosis. Balloon 100 of catheter 101 is inflatable. Balloon 100, of construction preferably similar to that of a standard angioplasty balloon catheter balloon, is typically inflatable by introduction of a pressurized fluid therein, in a manner well known in the art.

For use according to an embodiment of the present invention presented in FIG. 2, balloon 100 may be uninflated, or partially inflated, so that the presence of balloon 100 in blood vessel 150 does not seriously impede flow of blood within vessel 150 when vessel 150 is free of stenotic narrowing. Consequently, in the absence of stenosis-causing plaque, pressure readings taken by distal pressure sensor 120 will not differ substantially from pressure readings taken by proximal pressure sensor 110. This situation is presented by FIG. 2A.

FIG. 2B, in contrast, presents a situation in which balloon 100 is located in a region of vessel 150 wherein plaque deposits 160 have caused a narrowing of vessel 150. In this case, whichever pressure sensor (110 or 120) is situated “upstream”, closer to the source of blood flow (e.g., closer to the heart, if vessel 150 is an artery) will register a relatively higher blood pressure, and whichever sensor is situated “downstream”, further from the source of blood flow, will register a relatively lower blood pressure. If, for example vessel 150 is an artery and distal sensor 120 is closer than proximal sensor 110 to the heart, then distal sensor 120 will measure and report higher blood pressure than proximal sensor 110. This difference in blood pressure is caused wherever plaque deposits 160 impede free flow of blood between exterior wall 142 of balloon 100 and interior wall 152 of vessel 150. Reduction or elimination of blood flow between balloon 100 and interior wall 152 of vessel 150 results in a lower blood pressure measurement at the downstream sensor than at the upstream sensor.

Thus, significant differences between pressure readings from sensor 110 and sensor 120 indicate presence of a plaque deposit or other obstruction in vessel 150.

Uninflated or partially inflated balloon 100 may be passed gradually along a selected length of vessel 150, and readings from sensors 110 and 120 may be monitored at set intervals or continuously, so as to determine, at each position of balloon 100, whether significant differences in pressure between sensor 110 and sensor 120 have been detected.

The degree of inflation of balloon 100 best suited to the diagnostic procedure described above will depend on a variety of factors. Inflation of balloon 100 may be manipulated to optimize the differential sensitivity of pressure readings obtained from sensors 110 and 120. In one embodiment of the method here presented, balloon 100 may be passed several times along a selected length of vessel 150, with balloon 100 each time at a slightly increased expansion, so as to experimentally determine an optimal degree of expansion for a given selected length of a given vessel 150, that is, to experimentally determine the degree of expansion of balloon 100 which most clearly shows pressure differences between upstream and downstream pressure sensors at positions where stenosis is detected. Alternatively, balloon 100 may be expanded within a healthy segment of vessel 150 until a slight difference of pressure between the upstream and downstream pressure sensors is detected, and balloon 100 may then be caused to move along a selected length of vessel 150 so that a consistent set of pressure readings may be taken at that degree of expansion. In yet another alternative method, expansion and contraction of balloon 100 may be continuously adjusted (preferably under control of an automatic feedback mechanism) so as to maintain a constant ratio of pressure between upstream and downstream pressure sensors. In this case, the varying degree of expansion of balloon 100 required to maintain a constant pressure differential between upstream and downstream sensors over a selected length of vessel 150 can then be taken as a measure of the presence or absence of stenosis along that selected length of vessel 150.

In practice, a variety of clinical considerations, including the known or expected physiological profile of vessel 150 and the possible deleterious effects of prolonged interference of blood flow within vessel 150, will also contribute to a determination of the degree of expansion of balloon 100 most desirable for use in each particular clinical situation.

As an aid to recording and understanding the positions of balloon 100 at which a stenotic condition is detected, a proximal portion of catheter 101 may be provided with a graduated scale, indicating the length to which catheter 101 has penetrated into vessel 150, which scale can then be read by an operator when stenosis of vessel 150 is detected.

Alternatively, catheter 101 may be provided with one or more markers 170 (shown in FIG. 1) detectable under medical visualization modalities, which may then be used to photograph or otherwise record positions of balloon 100 at which a stenotic condition of vessel 150 is detected. Marker 170 may be a radio-opaque marker 172 visible under fluoroscopic or other x-ray examination. Marker 170 may also be an untrasound-detectable marker 174, detectable under ultrasound examination. Of course, the material composition of balloon 100 and the fluid selected to fill and inflate balloon 100, may themselves be visible under x-ray or ultrasound inspection, or under some alternate medical imaging modality, without need for special markers to render the position of balloon 100 visible.

Thus, obstruction of blood flow in a blood vessel at a selected location within that vessel may be detected by positioning balloon 100 at that selected location, (as shown in FIGS. 2A and 2B), and comparing pressure readings obtained from a pressure sensor distal to balloon 100 to pressure readings obtained from a pressure sensor proximal to balloon 100, and reporting obstruction of blood flow if a significant difference in pressure is detected. Typically, an operating physician will determine, based on clinical considerations, how much of a pressure difference should be considered “significant” in any particular case. Preferably, the diagnostic apparatus here described will be designed and constructed to report an obstruction when a detected pressure difference exceeds a pre-determined limit, which limit may be expressed either as an absolute pressure difference or as a percentage difference between the upstream and downstream pressure values.

Preferably, balloon 100 may be caused to pass continuously along a selected length of vessel 150, and pressure readings from sensors 110 and 120 may be monitored continuously to determine and report presence or absence of stenotic conditions, and degree of stenosis, along that selected length of vessel 150.

In a preferred embodiment, a plurality of pressure sensors 110 (110a, 110b, etc.) may be provided to enhance accuracy and reliability of pressure readings obtained by sensors 110. A data processing module may be used to receive and record pressure readings from multiple sensors 110, and average the result.

Similarly, a plurality of pressure sensors 120 (120a, 120b, etc.) may be provided to enhance accuracy and reliability of pressure readings obtained by sensors 120. A data processing module may be used to receive and record pressure readings from multiple sensors 120 and average the result.

Attention is now again directed to FIG. 1, and in particular to the use of pressure sensors 130 and 140 to detect and localize stenosis, and to distinguish standard plaque from vulnerable plaque.

Pressure sensor 130 is operable to measure and report pressure within expandable balloon 100. In a preferred embodiment of the present invention, balloon 100 is constructed similar to standard angioplasty balloons, in that balloon 100 is constructed of a semi-rigid material such as PVC or PET or nylon. Balloon 100 is inflatable when filled with a pressurized fluid which forces expansion of balloon 100. As is typical of most angioplasty balloon catheters in use today, in a preferred embodiment a fluid pressure of between 6 and 20 atmospheres is used to force expansion of balloon 100.

If balloon 100 is constructed with materials similar to those typically used for angioplasty balloons today, volumetric expansion of balloon 100 will be an approximately linear function of the pressure exerted by the fluid used to fill balloon 100. In any case, the degree and manner in which any given model of balloon 100 expands under pressure of an expansion fluid is measurable, and consequently a knowable predictable relationship will exist between changes in pressure within balloon 100, and consequent changes in balloon 100's external dimensions. Under the inflation pressures preferentially used (preferably between 6 and 20 atmospheres), pressures exerted on balloon 100 by walls 152 of blood vessel 150 will have only a negligible effect on the resultant dimensions of balloon 100 under a given inflation pressure, and can practically be ignored in calculating the external dimensions of balloon 100 under a selected inflation pressure.

Thus, if balloon 100 is connected to a controllable source of pressurized inflating fluid (such as a compressed gas, or a source of liquid under pressure), balloon 100 can be inflated to a desired external dimension, simply by inflating balloon 100 to a pressure calculated or observed to produce the required external dimension. Inflating balloon 100 to this desired inflation pressure can be accomplished by connecting balloon 100 to a pressurized fluid source in a system controlled by a feedback loop, wherein inflow of inflating fluid is made dependent on measuring a lower-then-desired pressure at pressure sensor 130 within balloon 100. We note, however, that for the present purpose, pressure sensor 130 need not necessarily be located within balloon 100. Pressure sensor 130 may equally well be located in some other portion of the inflation system, such as in a fluid conduit that is in fluid communication with inflatable balloon 100.

Indeed, the diagnostic method here described can alternatively be accomplished without use of pressure sensor 130. In an alternative embodiment, balloon 100 can be inflated to an unknown pressure, and the change in size of balloon 100 can be observed directly by accurate imaging of balloon 100 through use of an imaging modality such as a fluoroscope or an ultrasound system.

Thus, balloon 100 can be inflated to a selected size by controlled pressure inflation, or balloon 100 can be inflated to an arbitrary size and that size can then be measured.

In a presently preferred embodiment, balloon 100 is inflated up to a size at which external walls 142 of balloon 100 just touch inner walls 152 of vessel 150. Contact between walls 142 of balloon 100 and inner walls 152 of vessel 150 is detectable by sensors 140, which will begin to register an increase in pressure when such contact is established. Accurate dimensions of balloon 100 can then be calculated from a measure of balloon 100's internal pressure, readable from sensor 130, or alternatively balloon 100's size can be measured directly through use of an imaging modality.

In a preferred diagnostic use of this configuration, balloon 100 is caused to expand within vessel 150 until contact is established between balloon 100 and vessel walls 152, which surround balloon 100. External dimensions of balloon 100 are then calculated or measured as described above. The external dimensions of balloon 100, thus determined, constitutes a measure of the internal cross-section of vessel 150 at the location wherein these measurements are taken.

By progressively moving balloon 100 along a selected length of vessel 150, and, at a plurality of positions, inflating balloon 100 until contact with vessel walls 152 is established, measuring the size (e.g., the diameter) of balloon 100 at that point, then deflating balloon 100 sufficiently to enable to move it to a successive point along that selected length of vessel 150, it is possible to measure and report a series of size measurements which constitute an explicit dimensional profile of the interior dimensions of that selected length of vessel 150. This constitutes a method for detecting regions of obstruction of blood flow within a vessel, such as, for example, stenosis caused by presence of plaque within vessel 150.

In an additional preferred diagnostic use of the configuration described by FIGS. 1 and 2, this configuration may be used to diagnostically determine the type of plaque which is present within a blood vessel. Once contact has been established between balloon 100 and vessel walls 152 as described above, pressure within balloon 100 is further increased in a selected amount. Balloon 100 will then further expand to a calculatable and/or observable extent. This further expansion of balloon 100 will exert further pressure on pressure sensors 140, located between balloon 100 and vessel wall 152. As an expanded balloon 100 exerts pressure outward on vessel walls 152, walls 152 will exert a counter-pressure inward, which counter-pressure is measurable by sensors 140. Dividing a measure of the change in size of balloon 100 by a measure the change in pressure between walls 152 of vessel 150 and walls 142 of balloon 100 yields a measure of the elasticity of vessel 150 at that point.

This measure of the elasticity of vessel 150 constitutes a diagnostic tool for characterizing plaque within vessel 150. In particular, this measure of vessel wall elasticity enables to distinguish between standard plaque and vulnerable plaque. It has been clinically observed that what is known in the art as “standard plaque” or “stable plaque” is less flexible than a normal healthy vessel wall. It has further been clinically observed that what is known in the art as “vulnerable plaque” is more flexible than a normal healthy vessel wall. Consequently, by measuring the change in pressure exerted by vessel wall 152 on balloon 100, as balloon 100 undergoes a known amount of expansion, one can determine whether the change in pressure is similar to, greater, or lesser than what would be expected of a healthy vessel wall. A change in pressure similar to that which would be expected from a healthy vessel wall may be taken as a diagnostic indication that the vessel wall is in fact healthy at that point.

A measured pressure greater than that expected of a healthy vessel wall, on the other hand, indicates that that measured portion of the vessel wall is less flexible than normal. Thus, a measured pressure greater than that expected of a healthy vessel wall may be taken as a diagnostic indicator of the presence of standard plaque in the vessel at that position.

Similarly, if pressure measured by sensor 140 is less than that which would be expected of a healthy vessel wall, then the material of (or on) the vessel wall and in contact with balloon 100 at that point is shown to be more flexible than would be expected of a normal vessel wall. Such a condition may be taken as a diagnostic indicator of the presence of vulnerable plaque in the vessel at that point.

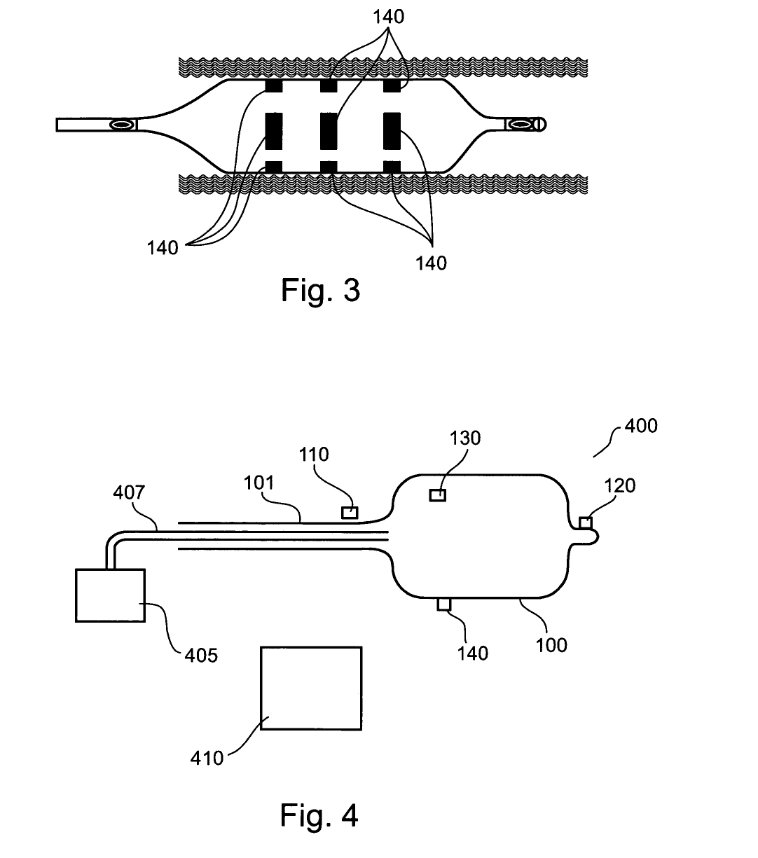

Attention is now drawn to FIG. 3, which is a simplified schematic of a preferred embodiment of the present invention, showing a preferred pattern of disposition of a plurality of pressure sensors 140. In a preferred embodiment, balloon 100 comprises a plurality of pressure sensors 140. This plurality of pressure sensors 140 are preferably arranged in concentric pattern around a circumference of balloon 100, or more preferably, in a plurality of concentric rings, as is shown in FIG. 3.

If balloon 100, comprising a plurality of sensors 140 arranged as shown in FIG. 3 is caused to expand to a known extent, changes in detected pressure at each of the plurality of sensors 140 can be independently measured. Asymetric contact between balloon 100 and vessel wall 152, indicating presence of plaque, and/or relative flexibility of local portions of wall 152, can thus be measured simultaneously at a plurality of points, thereby providing a high-resolution diagnostic image of the physical profile and condition of inner wall 152 of blood vessel 150.

Attention is now drawn to FIG. 4, which presents a system 400 for detecting and localizing obstructions in a vessel. System 400 comprises a balloon catheter 101 as described hereinabove, a balloon inflation system 405 which comprises means for controlled inflation of an inflatable balloon 100 of balloon catheter 101 by supply of a pressurized inflating fluid to balloon 100 through a pressurized inflation fluid delivery lumen 407. Preferably, balloon inflation system 405 comprises a feedback loop utilizing pressure data received from a pressure sensor 130 (which is operable to report pressure of an inflation fluid within balloon 100) to control delivery of pressurized inflation fluid to balloon 100.

System 400 further comprises a data processing module 410 operable to receive input from pressure sensors 110, 120, 130 and 140 of catheter 101, and further operable to analyze received pressure data according to principles of the present invention described hereinabove. In particular, data processing module 410 is operable to receive and to compare pressure reports from sensors 110 and 120, and to report a blood flow obstruction in a vessel when pressures detected by sensors 110 and 120 differ by more than a predetermined amount. Data processing module 410 is further operable to receive pressure measures reported by one or more pressure gauges 140, to compare these received pressure measures to predetermined expected “healthy” pressure values expected to received from healthy blood vessel tissues, and to report presence of standard plaque if received pressure measures are greater than the predetermined expected healthy pressure values, and to report presence of vulnerable plaque if received pressure measures are less than the predetermined expected healthy pressure values.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A balloon catheter operable to detect obstruction of blood flow within a blood vessel, comprising:
a. a controllably inflatable balloon;
b. a first pressure sensor operable to measure and report ambient pressure within said blood vessel at a position proximal to said balloon; and
c. a second pressure sensor operable to measure and report ambient pressure within said blood vessel at a position distal to said balloon.

2. The catheter of claim 1, wherein at least one of said first and second pressure sensors is operable to report pressure measurements to a data receiver by wire connection.

3. The catheter of claim 1, wherein at least one of said first and second pressure sensors is operable to report pressure measurements to a data receiver by wireless connection.

4. A method for detecting obstruction of blood flow within a blood vessel, comprising:
a. introducing into said blood vessel a balloon catheter which comprises
i. a balloon operable to be controllably inflated under pressure of a pressurized inflating fluid,
ii. a first pressure sensor operable to report ambient pressure within said blood vessel at a position proximal to said balloon, and
iii. a second pressure sensor operable to measure and report ambient pressure within said blood vessel at a position distal to said balloon;
b. obtaining a first pressure measurement of ambient pressure at said first sensor;
c. obtaining a second pressure measurement of ambient pressure at said second sensor; and
d. reporting obstruction of blood flow within said vessel if a significant difference is found to exist between said first pressure measurement and said second pressure measurement.

5. The method of claim 4, wherein a difference between said first pressure measurement and said second pressure measurement is treated as significant if said difference exceeds a predetermined value.

6. The method of claim 4, further comprising determining a position of a detected obstruction by determining a position of said balloon when a significant difference is found to exist between said first pressure measurement and said second pressure measurement.

7. The method of claim 6, further comprising determining said position of said balloon by determining a length of penetration of said catheter in said vessel by reading a graduated scale presented on a proximal portion of said catheter, which scale indicates a length to which said catheter has penetrated into said blood vessel.

8. The method of claim 6, further comprising determining said position of said balloon by utilizing an imaging modality to observe said catheter within said vessel.

9. The method of claim 6, further comprising determining said position of said balloon by utilizing an imaging modality to observe a marker on said catheter, which marker is visible under said imaging modality.

10. The method of claim 9, wherein said marker is radio-opaque.

11. The method of claim 10, wherein said imaging modality is a fluoroscope.

12. The method of claim 10, wherein said marker is visible under ultrasound scanning, and said imaging modality is an ultrasound system.

13. A method for measuring an internal dimension of a blood vessel, comprising:
a. introducing into said vessel a balloon catheter having a controllably expandable inflatable balloon and at least one first pressure sensor operable to report pressure between an outer wall of said balloon and an inner wall of said blood vessel;
b. expanding said balloon until contact is established between said outer wall of said balloon and said inner wall of said blood vessel, said contact being indicated by a rise in pressure reported by said at least one first pressure sensor; and
c. determining and reporting an external dimension of said balloon when said rise in pressure is detected, thereby measuring said internal dimension of said blood vessel.

14. The method of claim 13, wherein said external dimension of said balloon is determined by inspecting said balloon under an imaging modality.

15. The method of claim 14, wherein said imaging modality is an x-ray system.

16. The method of claim 14, wherein said imaging modality is a fluoroscope.

17. The method of claim 14, wherein said imaging modality is an ultrasound system.

18. The method of claim 13, wherein said external dimension of said balloon is determined by utilizing a second pressure sensor to measure pressure of an inflation fluid inflating said balloon, and calculating said external dimension as a function of said measured pressure of said inflation fluid as reported by said second pressure sensor.

19. The method of claim 18, wherein said calculation is based on known characteristics of expansibility of said balloon under varying conditions of pressure.

20. The method of claim 13, further comprising utilizing a plurality of said first pressure sensors.

21. The method of claim 20, wherein said plurality of first pressure sensors is arranged in a circumferential configuration on said balloon.

22. The method of claim 20, wherein said plurality of first pressure sensors is arranged in a plurality of circumferential configurations on said balloon.

23. A method for distinguishing between standard plaque and vulnerable plaque in a blood vessel, comprising:
a. introducing into said vessel a balloon catheter having a controllably expandable inflatable balloon and at least one first pressure sensor operable to report pressure between an outer wall of said balloon and an inner wall of said blood vessel;
b. expanding said balloon until contact is established between said outer wall of said balloon and said inner wall of said blood vessel, said contact being indicated by a detected rise in pressure reported by said at least one first pressure sensor;
c. further expanding said balloon to a controlled degree;
d. utilizing said at least one first pressure sensor to report pressure between said outer wall of said balloon and said inner wall of said blood vessel;
e. comparing said reported pressure to pressure values appropriate for healthy blood vessel wall tissues;
f. reporting presence of standard plaque if said reported pressure is greater than said values appropriate for healthy blood vessel tissues; and
g. reporting presence of vulnerable plaque if said reported pressure is less than said values appropriate for healthy blood vessel tissues.
